(19) United States
(12) Patent Application Publication
(10) Pub. No.: US 2024/0366832 A1
(43) Pub. Date: Nov. 7, 2024

(54) COMPOSITIONS AND RELATED METHODS

(71) Applicant: Boston Scientific Scimed, Inc., Maple Grove, MN (US)

(72) Inventors: Siavash Sam PARKHIDEH, Marlborough, MA (US); Nicolas BALL-JONES, Minneapolis, MN (US); Scott Edward CORBEIL, Litchfield, NH (US); Joseph Thomas DELANEY, Jr., Minneapolis, MN (US); Allison KUMARASENA, Wellesley, MA (US); Lauren Sfakis LYDECKER, Millbury, MA (US); Cristian PARISI, Boston, MA (US); Peijiang WANG, Brighton, MA (US); Dawn WINSOR-HINES, Northborough, MA (US)

(73) Assignee: Boston Scientific Scimed, Inc., Maple Grove, MN (US)

(21) Appl. No.: 18/654,634

(22) Filed: May 3, 2024

Related U.S. Application Data
(60) Provisional application No. 63/500,105, filed on May 4, 2023.

(51) Int. Cl. A61L 27/18

(52) U.S. Cl. CPC A61L 27/18; A61L 2400/06; A61L 2430/34

(57) ABSTRACT

Compositions useful in medical procedures are described, such as separating tissue layers, e.g., submucosal tissue layers, for removal. In some examples, the compositions may include a pharmaceutically acceptable carrier fluid and one or more biocompatible materials at least partially or completely dissolved or suspended in the pharmaceutically acceptable carrier fluid. The one or more biocompatible materials may be chosen from polymers, phospholipids, nanoclays, or combinations thereof. Advantageously, the compositions may be formulated as a solution, emulsion, gel, or suspension capable of being injected into tissue.

### CROSS-REFERENCE TO RELATED APPLICATIONS

This application claims the benefit of priority to U.S. Provisional Application No. 63/500,105, filed on May 4, 2023, which is incorporated by reference herein in its entirety.

### TECHNICAL FIELD

This disclosure relates generally to biocompatible and biodegradable materials useful for medical procedures, including endoscopic procedures.

### BACKGROUND

Colon cancer is the third most common cancer occurring in the United States. Cells in the colon mucosa have a risk of becoming cancerous, with increasing likelihood as humans age. Routine colonoscopies can identify precancerous or cancerous lesions. These lesions and other suspicious tissue can be excised endoscopically. Due to risk of perforation of the colon wall, steps are needed to reduce the risk of wall perforation during lesion removal. Prior methods have injected saline solution into a connective tissue layer (submucosa) of the colon to “lift” the lesion away from the rest of the tissue wall for removal. However, saline is rapidly absorbed by the body. Because this absorption time is shorter than many procedures, saline has only limited use.

### SUMMARY

The present disclosure includes compositions useful in medical procedures, e.g., for submucosal lifting. For example, the composition may include a pharmaceutically acceptable carrier fluid and one or more biocompatible materials at least partially or completely dissolved or suspended in the pharmaceutically acceptable carrier fluid. The pharmaceutically acceptable carrier fluid may include a buffer or saline solution, for example. The composition may be formulated as a solution, emulsion, gel, or suspension capable of being injected into tissue.

The one or more biocompatible materials may be chosen from polymers, phospholipids, nanoclays, or combinations thereof. In some examples, the one or more biocompatible materials may include polyethylene glycol (PEG) or derivative thereof, polyhydroxyethylmethacrylate, a poloxamer, 2-dimethylaminoethyl methacrylate, poly-L-lactic acid, polycaprolactone, a polyoxazoline, poly(lactic-co-glycolic acid), poly(N-isopropylacrylamide), polyvinyl alcohol, hyaluronic acid, alginate, pectin, agar, chia, chitosan, collagen, gelatin, dextran, heparin, a silk hydrogel, a peptide-based material, hydroxypropyl methylcellulose, methylcellulose, carboxymethylcellulose, or a combination thereof. In some examples, the one or more biocompatible materials may include PEG-succinimidyl glutarate (PEG-SG), PEG-succinimidyl succinate (PEG-SS), PEG-succinimidyl malonate (PEG-SM), or a combination thereof. The concentration of the one or more biocompatible materials may range from about 0.1% w/v to about 20% w/v.

The composition may further include one or more additives chosen from crosslinkers, photoactivatable materials, fluorescent materials, radiopaque materials, or combinations thereof. The crosslinker(s) may be chosen from tripolyphosphate or trilysine acetate. The composition may further include at least one photoactivatable material capable of being crosslinked with light, such that the composition may transition from a liquid to a gel. The composition may include a phospholipid, e.g., a phospholipid micelle and/or a phospholipid liposome. The composition may be formulated as an emulsion. The one or more biocompatible materials may be formulated as microparticles suspended in the pharmaceutically acceptable carrier fluid. In some examples, the microparticles may be suspended in a solution including the pharmaceutically acceptable carrier fluid and a polymer at least partially dissolved in the pharmaceutically acceptable carrier fluid. The composition may be in the form of a shear thinning solution or a gel, such as a hydrogel.

The composition may be used for removing a portion of mucosal tissue from a subject. In some examples, the composition may be administered to the subject by injection into submucosal tissue, and in some cases, where the portion of mucosal tissue includes a lesion.

According to some aspects of the present disclosure, a composition for submucosal lifting may include a pharmaceutically acceptable carrier fluid and one or more biocompatible materials. The one or more biocompatible materials may be chosen from polyethylene glycol (PEG), PEG-succinimidyl glutarate (PEG-SG), PEG-succinimidyl succinate (PEG-SS), PEG-succinimidyl malonate (PEG-SM), chitosan, gelatin, alginate, or a combination thereof. A concentration of the one or more biocompatible materials in the composition may range from about 0.1% w/v to about 20% w/v. In some examples, the composition may include tripolyphosphate or trilysine acetate. In some examples, the composition may include one or more additives chosen from crosslinkers, photoactivatable materials, fluorescent materials, radiopaque materials, or combinations thereof.

According to some aspects of the present disclosure, a composition for submucosal lifting may include a pharmaceutically acceptable carrier fluid, one or more biocompatible materials at least partially or completely dissolved or suspended in the pharmaceutically acceptable carrier fluid, and a crosslinker chosen from tripolyphosphate or trilysine acetate. The one or more biocompatible materials may be chosen from polyethylene glycol (PEG), PEG-succinimidyl glutarate (PEG-SG), PEG-succinimidyl succinate (PEG-SS), PEG-succinimidyl malonate (PEG-SM), chitosan, gelatin, alginate, or a combination thereof. In some examples, the composition may be formulated as a gel or microparticles.

### DETAILED DESCRIPTION

Particular aspects of the present disclosure are described in greater detail below. The terms and definitions provided herein control, if in conflict with terms and/or definitions incorporated by reference.

As used herein, the terms “comprises,” “comprising,” or any other variation thereof are intended to cover a non-exclusive inclusion, such that a process, method, composition, article, or apparatus that comprises a list of elements does not include only those elements, but may include other elements not expressly listed or inherent to such process, method, composition, article, or apparatus. The term “exemplary” is used in the sense of “example” rather than “ideal.”

As used herein, the singular forms “a,” “an,” and “the” include plural reference unless the context dictates otherwise. The terms “approximately” and “about” refer to being nearly the same as a referenced number or value. As used herein, the terms “approximately” and “about” should be understood to encompass ±5% of a specified amount or value. All ranges are understood to include endpoints, e.g., an amount ranging from 0.1 g to 10 g includes 0.1 g, 10 g, and all values between.

The present disclosure includes compositions useful in medical procedures, such as separating tissue layers, e.g., submucosal tissue layers, for removal. For example, the compositions herein may be useful in endoscopic mucosal resection (EMR) and endoscopic submucosal dissection (ESD) procedures. The compositions herein may be in the form of a solution, an emulsion, a suspension, gel (e.g., a hydrogel), microparticles (e.g., in a suspension), or a combination thereof.

The compositions herein may comprise one or more biocompatible materials and a pharmaceutically acceptable carrier fluid. For example, the biocompatible material(s) may be dissolved, dispersed, or suspended in a pharmaceutically acceptable carrier fluid. The biocompatible materials useful for the present disclosure include natural and synthetic polymers, including biopolymers, e.g., chemically-modified biopolymers. Biocompatible materials suitable for the compositions include, for example, polymers, phospholipids, nanoclays, and combinations thereof. Exemplary polymers include, e.g., polyethylene glycol (PEG) and derivatives thereof (e.g., PEG-succinimidyl glutarate (PEG-SG), PEG-succinimidyl succinate (PEG-SS), PEG-succinimidyl malonate (PEG-SM)), polyhydroxyethylmethacrylate (polyHEMA), thermogelling polymers such as poloxamers, 2-dimethylaminoethyl methacrylate (DMEMA), poly-L-lactic acid (PLLA), polycaprolactone (PCL), polyoxazolines, poly(lactic-co-glycolic acid) (PLGA), poly(N-isopropylacrylamide) (polyNIPAM), polyvinyl alcohol (PVA), hyaluronic acid, alginate (including, e.g., sodium alginate, calcium alginate, and barium alginate), pectin, agar, chia, chitosan, collagen, gelatin, dextran, heparin, silk hydrogels, peptide-based materials, hydroxypropyl methylcellulose (HPMC), methylcellulose, and carboxymethylcellulose. Exemplary phospholipids include, e.g., phospholipid micelles and phospholipid liposomes. Exemplary nanoclays include, for example, laponite (a smectic clay).

The pharmaceutically acceptable carrier fluid may comprise an aqueous liquid, e.g., a buffer such as phosphate buffer or borate buffer, or saline solution. In some examples herein, the composition comprises 0.9% w/v saline solution. The composition may be pH adjusted with a suitable acid (e.g., hydrochloric acid (HCl) or acetic acid) or base (e.g., sodium hydroxide (NaOH)).

The compositions herein may further comprise one or more additives, such as, e.g., crosslinkers, photoactivatable material(s), fluorescent material(s), radiopaque material(s), or combinations thereof. In some examples, the composition may comprise one or more crosslinkers, e.g., crosslinking the biocompatible material(s). Exemplary crosslinkers suitable for the compositions herein include, but are not limited to, tripolyphosphate (TPP), trilysine acetate (TLA), MMP-degradable peptides serving as crosslinkers (e.g., CGPQGIWGQC, CGPQGIAGQGCR, QPQGLAK), hydrolysable crosslinkers (e.g., organic compounds with ester linkages or R—O—Si—O—R′ groups), photocleavable crosslinkers (e.g., o-nitrobenzyl alcohol (ONB) or bis(maleimidoethyl) 3,3′-diselanediyldipropionoate (BMEDSeDP)), and combinations thereof. Exemplary photoactivatable materials include, but are not limited to, organic compounds comprising nitrobenzylalcohol functional groups, acrylate/methacrylate functional groups, and photoinitiators including, e.g., irgacure, and benzoyl peroxide. For example, the compositions herein may comprise benzoyl peroxide in a range of about 0.01% to about 5% w/v. Exemplary fluorescent materials include, but are not limited to, compounds chemically modified and/or mixed with fluorophores (e.g. green fluorescent protein (GFP), fluorescein isothiocyanate (FITC), cyanine dye, and/or combinations thereof). Exemplary radiopaque materials include, but are not limited to, compounds comprising radiopaque atoms, such as iodine, e.g., PEG and/or other polymers comprising radiopaque atoms, such as iodinated linear PEG or iodinated star PEG.

The compositions herein may be formulated for injection. Thus, for example, the composition may be injected in a submucosa layer, wherein the composition causes a space to form between the area of interest (e.g., lesion) to be excised and the underlying tissue. This space may facilitate removal of the area of interest, e.g., while minimizing damage to the underlying tissue. The following discussion includes examples of biocompatible materials useful for formulating compositions as solutions, emulsions, gels (e.g., hydrogels), microparticles, nanoparticles and suspensions, e.g., together with a pharmaceutically acceptable fluid, and optionally one or more additives.

### Solutions

In some aspects of the present disclosure, the composition comprises one or more biocompatible materials partially or completely dissolved in a pharmaceutically acceptable carrier fluid, e.g., forming a solution. The composition may be a shear thinning solution. The solution may be formulated for injection. Exemplary compositions formulated as a solution may comprise, for example, one or more polymers such as, e.g., PEG, polyHEMA, thermogelling polymer(s) such as poloxamer(s), DMEMA, PLLA, PCL, polyoxazoline(s), PLGA, polyNIPAM, hyaluronic acid, alginate (e.g., sodium alginate), pectin, agar, chia, collagen, gelatin, dextran, heparin, silk hydrogel(s), peptide-based material(s), HPMC, methylcellulose, or carboxymethylcellulose, derivatives thereof, and combinations thereof. The solution may comprise the biocompatible material(s) at a concentration ranging from about 0.1% w/v to about 20% w/v, such as about 1% w/v to about 15% w/v, or about 5% w/v to about 10% w/v. In some examples, the composition may comprise a combination of two or three biocompatible materials. In some examples, the composition may exhibit a thermally enhanced viscosity. For example, the composition may comprise one or more thermogelling polymers, such as polymers capable of thickening at body temperature (e.g., about 36.5° C. to 37.5° C.). For example, the composition may comprise a poloxamer and/or polyNIPAM.

As mentioned above, the composition may be formulated as a shear thinning solution. Shear forces of the composition may facilitate injection by a suitable injection device such as a syringe. Once injected, the composition may become viscous in the submucosa layer, retaining a bleb-like shape.

### Gels

The compositions herein may be formulated as a gel, e.g., capable of injection into tissue. The one or more biocompatible materials may be dispersed or at least partially or completely dissolved in a pharmaceutically acceptable carrier fluid, e.g., with an amount of crosslinking to form a gel. For example, the composition may be formulated as a loosely crosslinked hydrogel that is suitable for injection. In some examples, the biocompatible material(s) may comprise one or more polymers. For example, the composition may comprise PEG (including derivatives thereof such as PEG-SG, PEG-SS, PEG-SM, etc.), chitosan, gelatin, alginate (e.g., sodium alginate, calcium alginate, barium alginate, etc.), or a combination thereof. The biocompatible material(s) may be present at a concentration ranging of from about 0.1% w/v to about 20% w/v, such as about 1% w/v to about 15% w/v, for example about 5% w/v to about 10% w/v. Optionally, the composition may comprise one or more crosslinkers such as, e.g., TPP and/or TLA.

Exemplary compositions formulated as a gel include, but are not limited to, the following, with each component present in a concentration ranging from about 0.1% w/v to about 20% w/v, such as about 1% w/v to about 15% w/v, and more preferably about 5% w/v to about 10% w/v: (1) chitosan and TPP, (2) PEG-SG and TLA, (3) PEG-SS and TLA, (4) PEG-SM and TLA, (5) PEG-SG and chitosan, (6) PEG-SS and chitosan, (7) PEG-SM and chitosan, (8) gelatin, (9) alginate and calcium (Ca), (10) alginate and barium (Ba), and (11) combinations thereof.

Without being bound to any particular theory, the biocompatible material(s) may be crosslinked through non-covalent interactions, such as hydrogen bonds, van der Waals forces, and/or electrostatic interactions. As mentioned above, in some examples, the composition comprises one or more crosslinkers, e.g., to facilitate crosslinking.

Optionally, the gel may be processed or otherwise manufactured further into microparticles. For example, following formation of a gel, the gel may be formed into microparticles by pulverizing to create small particles.

The composition may be provided in a pre-gelled form, such as provided as separate composition in one or more containers to be mixed prior to injection. For example, the composition may be provided as components (e.g., a first biocompatible material and a second biocompatible material, or a biocompatible material and a crosslinker) in two or more separate containers to be mixed to form a gel suitable for injection. In some examples, the components may be provided in two or more syringes, which when simultaneously depressed may combine to form a gel, e.g., in a suitable delivery device (e.g., injection catheter) for administration to a subject.

### Emulsions

The compositions herein may be formulated as an emulsion capable of being injected, e.g., a shear-thinning emulsion. In some examples, the biocompatible material(s) of the emulsion comprise(s) a phospholipid, e.g., a phospholipid micelle, a phospholipid liposome, or a combinations thereof. Additionally or alternatively, the biocompatible material(s) of the emulsion may comprise a polymeric nanoemulsion, e.g., a nanoemulsion comprising PLLA, PCL, or a combination thereof, and/or the biocompatible material(s) may comprise a nanoclay. Exemplary nanoclays include, for example, laponite. The biocompatible material(s) may be present in the emulsion at a concentration ranging from about 0.1% w/v to about 10% w/v, such as about 1% w/v to about 4% w/v.

The composition may comprise two or more biocompatible materials at concentrations ranging from about 0.1% w/v to about 10% w/v, such as about 1% w/v to about 4% w/v. In at least one example, the composition in the form of an emulsion comprises a nanoclay at a concentration ranging from about 1% w/v to about 4% w/v.

### Microparticles

The compositions herein may be formulated as microparticles, e.g., a plurality of particles each having a size on the order of microns. The microparticles may be gel microparticles. As mentioned above, a gel may be formed by preparation of a gel and then processed to break the gel into a plurality of particles. Optionally, the composition may comprise microparticles and a pharmaceutically acceptable carrier, such as a buffer or saline solution. For example, the composition may comprise microparticles comprising one or more biocompatible materials, wherein the microparticles are suspended in a pharmaceutically acceptable carrier fluid, e.g., formulated as a suspension suitable for injection The biocompatible material(s) of the microparticles may comprise, for example, PEG (including derivatives thereof such as PEG-SG, PEG-SS, PEG-SM, etc.), chitosan, gelatin, alginate (including, e.g., sodium alginate, calcium alginate, barium alginate, etc.), or a combination thereof. Optionally, the microparticles may comprise one or more crosslinkers (e.g., TPP, TLA, or combinations thereof), facilitating formation of a gel of the particles.

The biocompatible material(s) may be present in the microparticles at a concentration ranging from about 0.1% w/v to about 20% w/v, such as about 1% w/v to about 15% w/v, e.g., about 5% w/v to about 10% w/v. Exemplary microparticles according to some aspects of the present disclosure may comprise the following, e.g., with each component at a concentration ranging from about 0.1% w/v to about 20% w/v, such as about 1% w/v to about 15% w/v, e.g., about 5% w/v to about 10% w/v: (1) chitosan and TPP, (2) PEG-SG and TLA, (3) PEG-SS and TLA, (4) PEG-SM and TLA, (5) PEG-SG and chitosan, (6) PEG-SS and chitosan, (7) PEG-SM and chitosan, (8) gelatin, (9) alginate and Ca, (10) alginate and Ba, (11) PVA, and (12) combinations thereof.

In some examples, the composition comprises microparticles combined with a solution comprising a pharmaceutically acceptable carrier fluid and one or more biocompatible materials chosen from PEG (and derivatives thereof), polyHEMA, thermogelling polymers such as poloxamers, DMEMA, PLLA, PCL, polyoxazolines, PLGA, hyaluronic acid, alginate (e.g., sodium alginate, calcium alginate, barium alginate) pectin, agar, chia, collagen, gelatin, dextran, heparin, silk hydrogels, peptide-based materials, HPMC, methylcellulose, carboxymethylcellulose, and combinations thereof. The biocompatible material(s) may be present in the composition at a concentration ranging from about 5% w/v to about 50% w/v, such as about 15% w/v to about 35% w/v, for example about 20% w/v to about 30% w/v.

It will be appreciated that the compositions herein (e.g., solutions, emulsions, gels, suspensions, microparticles, etc.) may be formulated with the desired viscosity, shear strength, degradability, and ability to lift/separate tissue layers to suit a particular medical procedure and needs of a patient. For example, the composition may have a viscosity of at least about 0.001 Pas, such as greater than about 0.1 Pa·s. As mentioned above, any of the compositions herein may comprise one or more additives, which may contribute to and/or impart desired properties. Exemplary additives include, for example, crosslinkers, photoactivatable materials, fluorescent materials, radiopaque materials.

Crosslinkers may be selected depending on the desired degree of crosslinking and/or desired degradability of the composition. While the compositions herein are generally biodegradable, one or more crosslinkers may be added to affect degradability, e.g., via engineered degradability provided by the crosslinker(s). The compositions herein may comprise, for example, one or more crosslinkers chosen from MMP-degradable crosslinkers (e.g., CGPQGIWGQC, CGPQGIAGQGCR, QPQGLAK), hydrolysable crosslinkers (e.g., polymers comprising ester linkages or R—O—Si—O—R′ groups), photocleavable crosslinkers (e.g., o-nitrobenzyl alcohol (ONB) or bis(maleimidoethyl) 3,3′-diselanediyldipropionoate (BMEDSeDP)), or combinations thereof. The crosslinkers may be designed to be degraded by the body's natural mechanisms (e.g., MMPs, hydrolysis) or external stimuli (e.g., near IR light, injection of enzymes). Crosslinkers may provide weak points in a composition, e.g., to provide tuneability of the degradation rate and forced activation of the composition.

Photoactivatable materials (e.g., materials that can be crosslinked with light) may be useful also to impart desired properties such as viscosity, shear strength, and/or degradability. The compositions herein may comprise one or more photoactivatable materials such as polymers comprising nitrobenzylalcohol functional groups, acrylate/methacrylate functional groups, and/or a photoinitiator such as irgacure or benzoyl peroxide. The photoactivatable material(s) may be capable of crosslinking when exposed to light, and increase in viscosity (e.g., transition the composition from a liquid to a gel, e.g., a relatively firm gel). The compositions herein possess properties and/or characteristics useful for improved ease of injection and for bleb formation, function, and/or maintenance.

Fluorescent materials useful for the present disclosure include fluorophores (e.g., compounds that comprise green fluorescent protein (GFP), fluorescein isothiocyanate (FITC), or cyanine dye, for example). The fluorescent materials may be useful for in situ visualization, such as bleb visualization and/or post-resection identification of composition remnants.

Radiopaque materials useful for the present disclosure include compounds that include radiopaque atoms and/or functionalized with moieties comprising radiopaque atoms. Suitable radiopaque materials may comprise, for example, iodine (e.g., PEG comprising moieties containing radiopaque atoms, such as iodinated linear PEG or iodinated star PEG), although the compositions are not limited to iodine. Radiopaque materials may be useful for post procedure visualization of the composition.

The compositions herein may be useful in various medical procedures that include removal of select tissue, e.g., to separate a portion of tissue from underlying tissue layers to minimize damage to the underlying tissue layers. Such medical procedures may include endoscopic procedures to lift a cellular growth (e.g., a polyp or lesion) from the mucosa layer of tissue. Such tissue may be tissue of the gastrointestinal tract, e.g., the colon. As discussed above, when a sufficient and/or predetermined amount of a composition is injected into the submucosa layer, the viscosity of the composition and/or the structural integrity of the composition may be capable of causing the outermost portion of tissue relative to the injection site to physically lift away from the underlying mucosa layer. In addition to providing physical lift, the compositions herein may be capable of providing a sustained lift extending over a prolonged period of time, e.g., suitable for a medical professional to complete removal of the select portion of tissue.

Other aspects of the present disclosure will be apparent to those skilled in the art from consideration of the specification and practice of the examples disclosed herein. It is intended that the specification and examples be considered as exemplary only, with a true scope and spirit of the present disclosure being indicated by the following claims.

We claim:

1. A composition for submucosal lifting, the composition comprising:
a pharmaceutically acceptable carrier fluid; and
one or more biocompatible materials at least partially or completely dissolved or suspended in the pharmaceutically acceptable carrier fluid, the one or more biocompatible materials being chosen from polymers, phospholipids, nanoclays, or combinations thereof;
wherein the composition is formulated as a solution, emulsion, gel, or suspension capable of being injected into tissue.

2. The composition of claim 1, wherein the one or more biocompatible materials comprises polyethylene glycol (PEG) or derivative thereof, polyhydroxyethylmethacrylate, a poloxamer, 2-dimethylaminoethyl methacrylate, poly-L-lactic acid, polycaprolactone, a polyoxazoline, poly(lactic-co-glycolic acid), poly(N-isopropylacrylamide), polyvinyl alcohol, hyaluronic acid, alginate, pectin, agar, chia, chitosan, collagen, gelatin, dextran, heparin, a silk hydrogel, a peptide-based material, hydroxypropyl methylcellulose, methylcellulose, carboxymethylcellulose, or a combination thereof.

3. The composition of claim 2, wherein the one or more biocompatible materials comprises PEG-succinimidyl glutarate (PEG-SG), PEG-succinimidyl succinate (PEG-SS), PEG-succinimidyl malonate (PEG-SM), or a combination thereof.

4. The composition of claim 1, wherein the pharmaceutically acceptable carrier fluid comprises a buffer or saline solution.

5. The composition of claim 1, wherein a concentration of the one or more biocompatible materials in the composition ranges from about 0.1% w/v to about 20% w/v.

6. The composition of claim 1, wherein the composition further comprises:
one or more additives chosen from crosslinkers, photoactivatable materials, fluorescent materials, radiopaque materials, or combinations thereof; and/or
one or more crosslinkers chosen from tripolyphosphate or trilysine acetate.

7. The composition of claim 1, wherein the composition further comprises at least one photoactivatable material capable of being crosslinked with light, such that the composition transitions from a liquid to a gel.

8. The composition of claim 1, wherein the composition comprises a phospholipid.

9. The composition of claim 8, wherein the composition comprises a phospholipid micelle or a phospholipid liposome.

10. The composition of claim 1, wherein the composition is formulated as an emulsion.

11. The composition of claim 1, wherein the one or more biocompatible materials are formulated as microparticles suspended in the pharmaceutically acceptable carrier fluid.

12. The composition of claim 11, wherein the microparticles are suspended in a solution comprising the pharmaceutically acceptable carrier fluid and a polymer at least partially dissolved in the pharmaceutically acceptable carrier fluid.

13. The composition of claim 1, wherein the composition is in the form of a shear thinning solution or a gel.

14. A method for removing a portion of mucosal tissue from a subject, the method comprising injecting the composition of claim 1 into submucosal tissue of the subject.

15. The method of claim 14, wherein the portion of mucosal tissue includes a lesion.

16. A composition for submucosal lifting, the composition comprising:
a pharmaceutically acceptable carrier fluid; and
one or more biocompatible materials being chosen from polyethylene glycol (PEG), PEG-succinimidyl glutarate (PEG-SG), PEG-succinimidyl succinate (PEG-SS), PEG-succinimidyl malonate (PEG-SM), chitosan, gelatin, alginate, or a combination thereof,
wherein a concentration of the one or more biocompatible materials in the composition ranges from about 0.1% w/v to about 20% w/v.

17. The composition of claim 16, wherein the composition further comprises tripolyphosphate or trilysine acetate.

18. The composition of claim 16, wherein the composition further comprises one or more additives chosen from crosslinkers, photoactivatable materials, fluorescent materials, radiopaque materials, or combinations thereof.

19. A composition for submucosal lifting, the composition comprising:
a pharmaceutically acceptable carrier fluid;
one or more biocompatible materials at least partially or completely dissolved or suspended in the pharmaceutically acceptable carrier fluid, the one or more biocompatible materials being chosen from polyethylene glycol (PEG), PEG-succinimidyl glutarate (PEG-SG), PEG-succinimidyl succinate (PEG-SS), PEG-succinimidyl malonate (PEG-SM), chitosan, gelatin, alginate, or a combination thereof; and
a crosslinker chosen from tripolyphosphate or trilysine acetate.

20. The composition of claim 19, wherein the composition is formulated as a gel or microparticles.
